(19) United States
(12) Patent Application Publication
(10) Pub. No.: US 2006/0184066 A1
(43) Pub. Date: Aug. 17, 2006

(54) Method for aiding stent-assisted coiling of intracranial aneurysms by virtual parent artery reconstruction

(75) Inventors: Christof Karmonik, Houston, TX (US); Michel E. Mawad, Houston, TX (US)

Correspondence Address: TIM HEADLEY, GARDERE WYNNE SEWELL LLP, 1000 LOUISIANA, SUITE 3400, HOUSTON, TX, 77002, US

(73) Assignee: Baylor College of Medicine, Houston, TX (US)

(21) Appl. No.: 11/058,131

(22) Filed: Feb. 15, 2005

(51) Int. Cl. A61B 5/103

(52) U.S. Cl. 600/587

(57) ABSTRACT

A method of creating a surface model of an intracranial aneurysm in an artery having a lumen, the aneurysm having a neck and a dome and the virtual reconstruction of the parent artery across the lateral extension of the aneurysm neck. The method includes the steps of: determining a center and radius of the artery over the lateral extension of the aneurysm; determining the boundary points that mark the boundary between the aneurysm neck and the artery; determining the angle of the aneurysm neck with respect to the artery, for various cross sections of the neck; determining the length of the neck; determining the height of the dome; estimating the area of the neck; and creating the surface model of the intracranial aneurysm in the artery, using the results from the previous steps.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A “SEQUENTIAL LISTING,” A TABLE, OR A COMPUTER PROGRAM LISTING APPENDIX SUBMITTED ON A COMPACT DISC

This application includes a computer program listing appendix, pursuant to 37 CFR 1.96, contained on a compact disc, which is incorporated fully into this application by this reference.

The compact disc is labeled as follows:
Inventors: Christof Karmonik & Michel Mawad
Title: Method For Visualization And Characterization Of Intracranial Aneurysms
Attorney docket number: 124169-1015
Creation date of the compact disc: Jun. 19, 2003

The compact disc contains the following files in ASCII file format:

| File Name | File size | Creation Date |
|---|---|---|
| Sourcecode.txt | 57 kb | Sep. 30, 2002 |

### BACKGROUND OF THE INVENTION

1. Field of the Invention

The present invention relates to an algorithm for actual and virtual three-dimensional reconstruction. In particular, the present invention relates to an algorithm for reconstructing actual and virtual three-dimensional images of an anatomical structure using images acquired with any medical 3D imaging method. The application of this algorithm is illustrated by, but not limited to, digital subtraction angiography.

An arterial aneurysm is a localized enlargement of an artery. Cerebral saccular aneurysms, the most common variety of intracranial aneurysms (aneurysms of brain vessels), are “balloon-like” protrusions of intracranial arteries characterized by an opening (“neck”) that feeds into an enlarged capsular structure (“dome”).

The rupture of an intracranial aneurysm is a catastrophic event that may potentially lead to severe disability or death. Even after treatment, there is a possibility for certain aneurysms to rupture. Considerable research efforts therefore focus on developing a deeper understanding of the geometry, hemodynamics and morphologic changes in aneurysms to optimize treatment options and improve outcomes. Geometrical factors such as morphology, neck size and dome-to-neck ratio particularly impact outcomes in endovascular treatment.

The current standard of care for treatment of intracranial aneurysms is surgical intervention. The goal of treatment is to reconstruct the artery segment across the neck of the aneurysm, thereby eradicating the aneurysm from normal circulation without compromising any of the adjacent vessels or small perforating branches of these vessels. This is currently done by surgical clipping of the neck of the aneurysm or by filling the dome of the aneurysm with material (e.g. metal coils, liquid, etc.) so that the blood coagulates. Thus, prior to treatment, it is necessary to characterize the anatomy of the neck of the aneurysm and the surrounding blood vessels. The endovascular therapist uses this characterization of the geometry and morphology of the aneurysm in treatment planning.

2. Description of Related Art

The following references form part of the related art, and are all incorporated into this patent by this reference:

United States Patents:

U.S. Pat. No. 6,714,661 Method and system for customizing facial feature tracking using precise landmark finding on a neutral face image
U.S. Pat. No. 6,684,098 Versatile stereotactic device and methods of use
U.S. Pat. No. 6,661,869 Image reconstruction using multiple X-ray projections
U.S. Pat. No. 6,587,541 Three dimensional image reconstruction from single plane x-ray fluorograms
U.S. Pat. No. 6,580,811 Wavelet-based facial motion capture for avatar animation
U.S. Pat. No. 6,563,950 Labeled bunch graphs for image analysis
U.S. Pat. No. 6,510,241 Process for reconstructing a three-dimensional image of an object
U.S. Pat. No. 6,473,488 Three dimensional image reconstruction from single plane X-ray fluorograms
U.S. Pat. No. 6,470,070 Image reconstruction using multiple X-ray projections
U.S. Pat. No. 6,466,695 Procedure for automatic analysis of images and image sequences based on two-dimensional shape primitives
U.S. Pat. No. 6,370,417 Method for positioning a catheter in a vessel, and device for implementing the method
U.S. Pat. No. 6,356,659 Labeled bunch graphs for image analysis
U.S. Pat. No. 6,317,621 Method and device for catheter navigation in three-dimensional vascular tree exposures
U.S. Pat. No. 6,301,370 Face recognition from video images
U.S. Pat. No. 6,272,231 Wavelet-based facial motion capture for avatar animation
U.S. Pat. No. 6,222,939 Labeled bunch graphs for image analysis
U.S. Pat. No. 6,080,164 Versatile stereotactic device
U.S. Pat. No. 6,041,097 Method and apparatus for acquiring volumetric image data using flat panel matrix image receptor
U.S. Pat. No. 5,588,033: “Method And Apparatus For Three Dimensional Image Reconstruction From Multiple Stereotactic Or Isocentric Backprojections”.
   United States Published Patent Applications:
20020193686 Methods and systems for performing medical procedures with reference to projective image and with respect to pre-stored images

The conventional technology used to visualize the geometry of intracranial aneurysms creates mainly two-dimensional surface models or two-dimensional projections of 3D models of the artery and the aneurysm based on images acquired with digital subtraction angiography (DSA). These surface models are either opaque or semitransparent. Conventional technology does not provide a means to visualize the reconstructed artery segment before treatment occurs, nor is there any known technology that attempts to do so.

A further shortcoming of conventional technology is its use of two-dimensional projection images to determine the dome height and the neck length of the aneurysm. This is potentially misleading, as these determinations are not made using all the three-dimensional (3D) data available.

What is needed is a method to facilitate treatment successes by supplying endovascular therapists with an enhanced reconstruction of the geometry and morphology of intracranial aneurysms for purposes of pretreatment planning.

Furthermore, a method is needed that will truly provide three-dimensional parameters that can be correlated with treatment outcomes.

The availability of stents designed specifically for use in the intracranial vasculature has increased the use of stent-assisted coiling for treatment of wide necked and complex intracranial aneurysms. Both because of the complex relationships between many aneurysms and their parent artery and the lack of an ability to visualize a stent fluoroscopically, it is often difficult to achieve a working projection adequate to assure that coils or loops of coils are not confined behind a stent and are not herniating through a stent cell so as to compromise the parent artery lumen. Because of this, treating physicians often find it necessary to use balloon neck protection as an adjunct to stent assisted coiling. As this adds complexity to the procedure, what is needed is a way to facilitate the ability to understand, during treatment, the location of coils as they are placed and detached into an aneurysm.

While commercially available 3D-DSA post-processing techniques allow the depiction of the external anatomical features of intracranial aneurysms, they fall short of being able to depict clearly the topography of an aneurysm ostium to its parent artery. What is needed is a pre-treatment post-processing algorithm that will provide a virtual image of the full extent of a stent in the parent artery, and, more specifically, an algorithm that allows insertion and visualization of a virtual stent.

### BRIEF SUMMARY OF THE INVENTION

The present invention is a method of creating a surface model of an intracranial aneurysm in an artery having a lumen, the aneurysm having a neck and a dome, the method comprising the steps of:
a. determining a boundary between the lumen and the surrounding tissue;
b. determining a center and radius of the artery over the lateral extension of the aneurysm;
c. determining the boundary points that mark the boundary between the aneurysm neck and the artery;
d. determining the angle of the aneurysm neck with respect to the artery, for various cross sections of the neck;
e. determining the length of the neck;
f. determining the height of the dome;
g. estimating the area of the neck; and
h. creating the 3D surface model of the intracranial aneurysm in the artery, using the results from the previous steps.

In an alternate embodiment of the present invention, it is a method to allow full visualization of a virtual stent deployed across an aneurysm ostium, including the steps of:
a. manually clipping a 3D-DSA data to obtain a volume of interest containing the aneurysm and proximal and distal segments of a healthy parent artery;
b. computing the centerline of the normal segments of the parent artery, proximal and distal to the aneurysm, using image post-processing skeletonization algorithms;
c. from these centerline segments, interpolating the centerline of the parent artery across the aneurysm ostium;
d. obtaining a set of contiguous 2D cross sections (cut planes) (approx. 0.1 mm thickness) containing the entire volume of the normal parent artery segments and the aneurysm, and oriented perpendicular to the interpolated centerline;
e. for each cross section containing a portion of the aneurysm, linearly interpolating the corresponding radius of the virtual parent artery, using the radii measured at the normal proximal and distal segments of the parent artery; and
f. projecting the resulting reconstruction for analysis in three different views: a) as a series of 2D cross sections, b) as a 3D cut surface reconstruction (clipped by a cut plane so as to allow inspection of the inside of the aneurysm), and c) as a 3D surface rendered volume.

The method of the present invention offers an enhanced ability to visualize and to understand the complex relationships between an aneurysm and its parent artery, thus functioning as a pre-treatment planning aid.

The ability to visualize a virtual stent prior to treatment improves the ability to monitor coil deposition during treatment, because it provides the operator with a priori knowledge both about the relationships between the aneurysm ostium and the parent artery, and the location of stent boundaries that can not be visualized directly during treatment.

### BRIEF DESCRIPTION OF THE FIGURES

The following figures are included to demonstrate specific features and advantages of the above-mentioned invention. These drawings are by way of example, and not by way of limitation. They use like references to indicate similar elements.

FIG. 1 illustrates a three-dimensional MIP reconstruction of an aneurysm with MPR cross section (shown in yellow) perpendicular to the axis of the artery.

FIG. 2 illustrates three views of an aneurysm, including a cross section, a threshold image and an extracted boundary set point.

FIG. 3 is a plot of the boundary points of the aneurysm neck derived by the algorithm.

FIG. 4 illustrates a reconstructed artery superimposed onto an aneurysmal artery.

FIG. 5 is a plot of the aneurysm neck area versus maximum neck angle.

FIG. 6 is a plot of aneurysm neck area versus aneurysm neck length.

FIG. 7 depicts three-dimensional surface models of selected aneurysms.

FIG. 8 is a graphical representation of the maximum neck angle distribution.

FIG. 9a is a flow chart summarizing the steps of the method of the invention.

FIG. 9b is a flow chart providing the details for the the eight steps mentioned in FIG. 9a.

FIG. 9c is a flow chart summarizing an alternate method of the steps of the invention.

FIG. 10 are 2D DSA pre-treatment projection images for case 1: a) AP view, b) lateral view).

FIG. 11 is a 3D DSA surface volume reconstruction for case 1.

FIG. 12 is a) a selected cut-plane section, b) a cut-surface volume reconstruction and c) a 3D DSA surface volume reconstruction with virtual stent for case 1.

FIG. 13 are 2D DSA post-treatment projection images for case 1: a) AP view, b) lateral view).

FIG. 14 are 2D DSA pre-treatment projection images for case 2: a) AP view, b) lateral view).

FIG. 15 is a) a selected cut-plane section and b) a cut-surface volume reconstruction for case 2.

FIG. 16 are 2D post-treatment projection images for case 2: a) DSA AP view, b) DSA lateral view), c) native lateral view.

### DETAILED DESCRIPTION OF THE INVENTION

The preferred embodiment of the method of the present invention is a computer program, written in Java and C++ programming languages. The computer program listing is attached in an appendix, pursuant to 37 CFR 1.96, contained on a compact disc, which is incorporated fully into this patent by this reference. The computer program listing is source code, and includes three parts. The first part is an implementing file for the aneurysm visualization algorithm. The second part is a file implementing the advanced neck finding algorithm. The third part is a file implementing the single value decomposition algorithm.

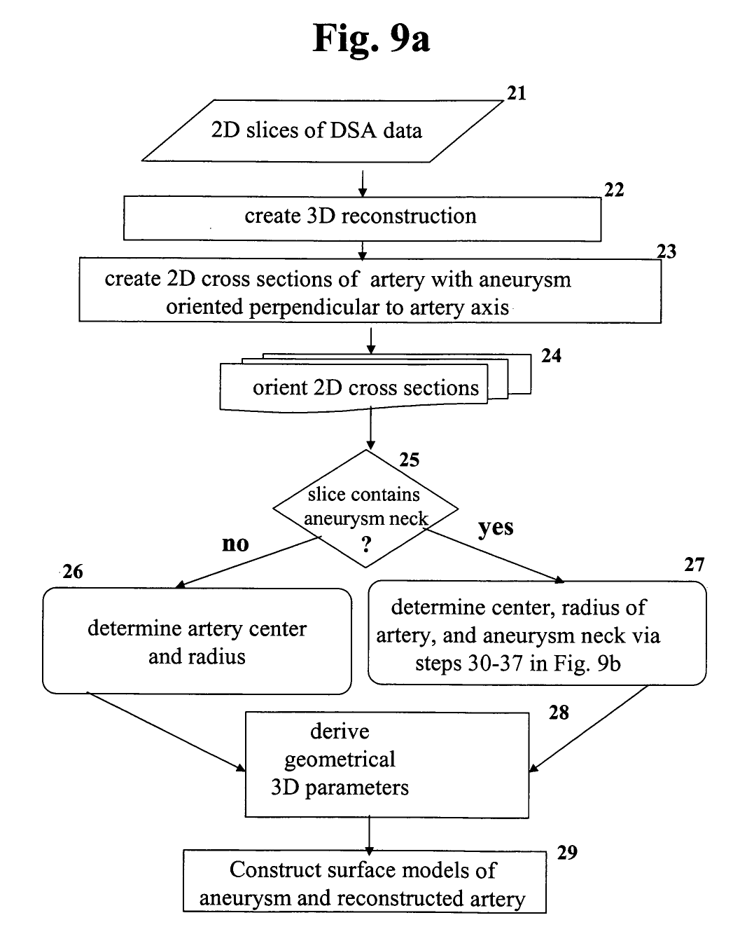

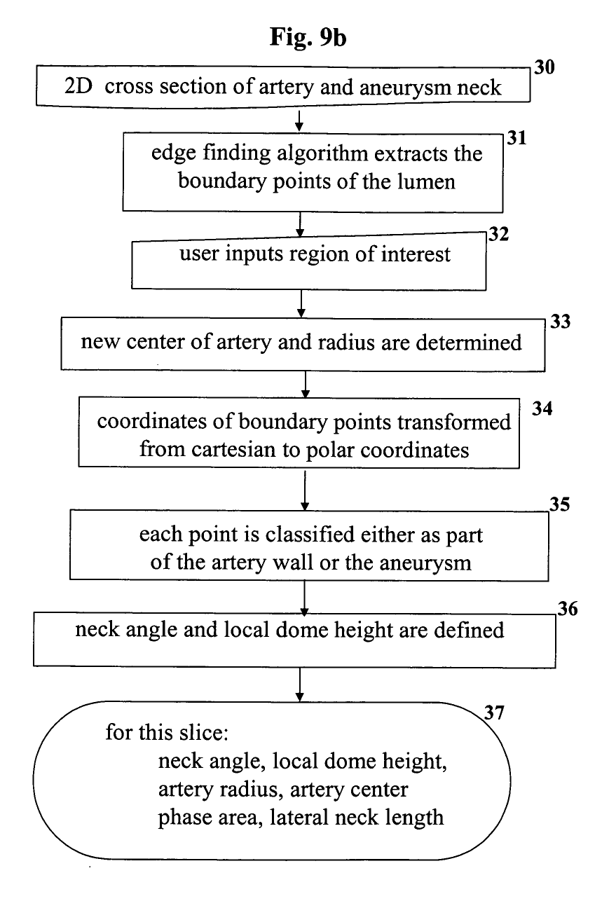

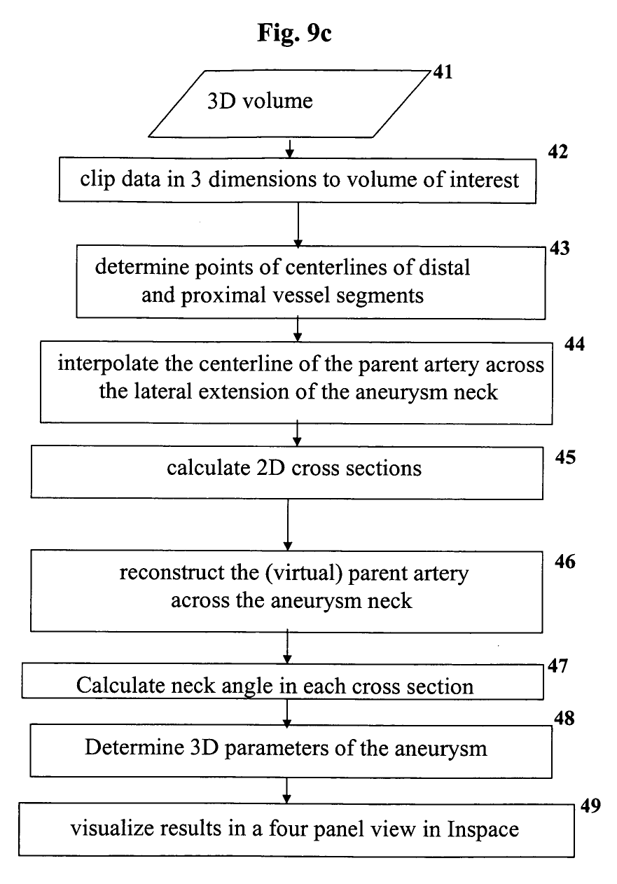

For ease of explanation the method of the invention is divided into five general segments. The method of the invention in its entirety is illustrated in the flow charts in FIGS. 9a and 9b.

1. Determining the Lumen Boundary

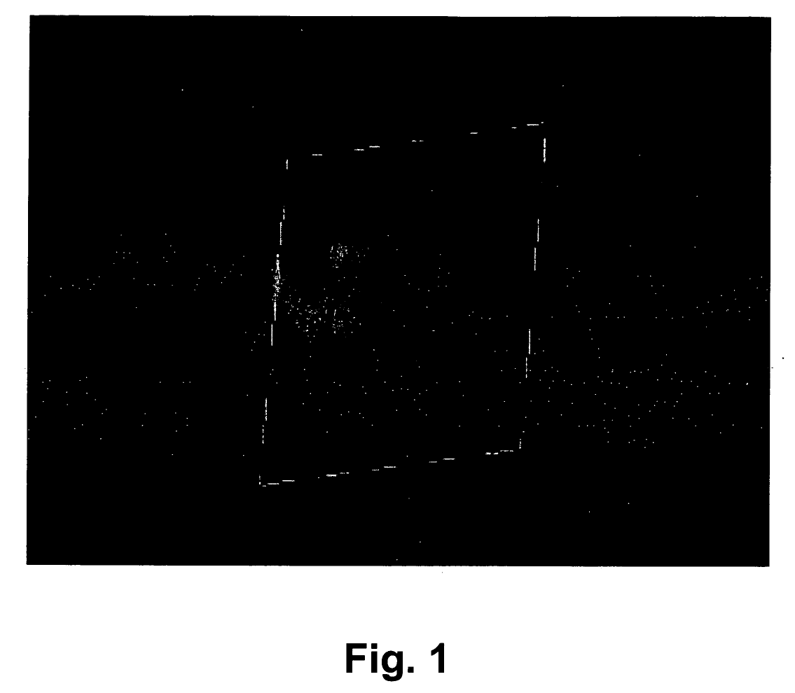

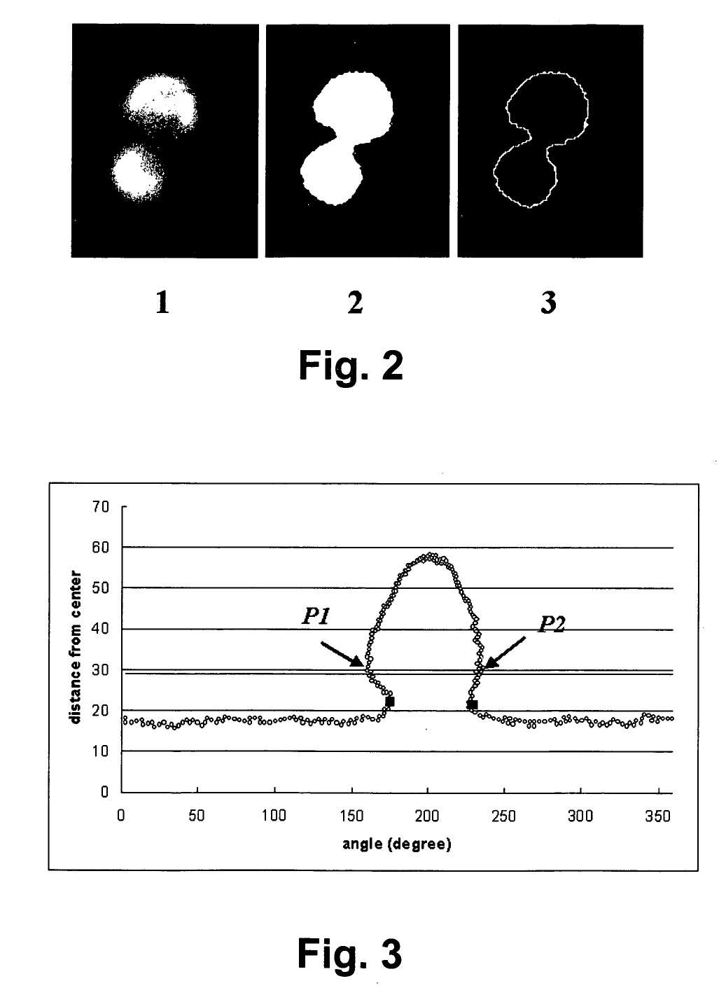

Referring now to FIG. 9a, in step 21 the image data is constructed for each aneurysm, the image data including a set of contiguous 2D DSA images. In step 22 the Merge eFilm Workstation software constructs a 3D maximum intensity projection (MIP) from the 3D DSA images to visualize the orientation of the parent artery and the aneurysm. In step 23 the multiple projection reconstruction (MPR) feature of eFilm creates a set of 2D cross sections (0.1 mm thickness) oriented approximately perpendicular to the axis of the parent artery (yellow square in FIG. 1), over the whole lateral extension of the aneurysm. The choice of the number of cross sections is made so that at least one of the cross sections (proximal or distal to the aneurysm neck) contains only the artery. If necessary, in step 24 the order of the slices is reversed so that this slice is the first in the set (see also step 30). The creation of the cross sections is not limited to the eFilm software, but can also be carried out with any other image processing program as long as it provides the true pixel size for these cross sections. These images are then imported into ImageJ for further processing.

All images are thresholded to obtain a sharp boundary between the lumen (the interior open part of the artery) and the surrounding tissue that makes up the wall of the artery. Presently, this thresholding is done by visual inspection, but other methods which use more sophisticated algorithms (e.g. identifying pixels of the lumen that have a grayscale value as the average of the minimum and maxium grayscale value in the image) are also feasible. In step 31 the modified ImageJ Wand tracing tool semi-automatically extracts the boundary points of the lumen from the thresholded image (see FIG. 9b).

2. Determination of Center and Radius of the Parent Artery

In step 25 the 2D cross section slices are sorted according to whether or not the slices contain the aneurysm neck. In step 26, for the first cross section containing only the artery, the center of this artery (x0,y0) is found as the center of mass of the boundary points. The radius R0 of the artery is calculated as the average distance between the boundary points and this center.

For the remaining cross section slices, a novel semi-automated algorithm is used to find the vessel center. It is based on the assumption that the coordinates for the center point exhibit only small changes for consecutive cross sections. In step 32 the user first defines a region of interest (ROI). This ROI contains only boundary points belonging to the arterial wall. All other boundary points outside this ROI are ignored in the further steps of the algorithm.

A reduced set of these boundary points is then identified within a distance of R0*(1+δ) from the coordinates (x0,y0) (the center point for the first slice). Only these boundary points are taken into account to calculate the artery center and the artery radius for this cross section. This step 32 accounts for variation in the vessel shape and the parameter δ and can be adjusted by the user to improve the outcome of the calculation.

A single value decomposition (SVD) algorithm is then utilized to find the coordinates (xs,ys) of a point which is located at minimum distance to the reduced set of boundary points. This point is the best approximation to the center of the artery assuming a circular shape of the arterial wall. In step 33 the radius Rs of the artery is determined as the average distance of this center to the reduced set of boundary points.

Iteratively applied to all remaining cross sections, this algorithm determines center and radius of the artery over the lateral extension of the aneurysm.

3. Determination of the Aneurysm Neck Angle

The following algorithm is used to determine the angle of the aneurysm neck in each cross section. In step 34, the coordinates of all the boundary points are transformed from Cartesian coordinates to polar coordinates (x,y)−>(d,ζ) with the center of the artery (xs,ys) as origin, so that d is the distance of a boundary point from this center and the polar angle ζ is the angle between the y axis and the line connecting the center and the boundary point. The boundary point set is ordered and stored in a circular buffer, so that point i+1 is the topological neighbor of point i (and point 0 is the neighbor of point N-1).

Traversing the ordered boundary point set, the distance of each boundary point is tested if it is smaller than Rs*(1+ε). (The parameter ε is introduced to account for the variation in vessel shape. A value of 0.3 for ε(i.e. 30%) was found to be suitable for all investigated cases). The algorithm stops if it finds a point (labeled P1) with a distance larger than this value (see FIG. 9b).

Depending on the parameter ε, the search algorithm tends to overestimate the angle marking the beginning of the aneurysm neck. Therefore, in a second step, the search algorithm starts from P1 traversing the boundary point set in descending order as long as the angle ζ decreases to find the correct first boundary point of the aneurysm neck. The algorithm then continues to traverse the boundary point set in ascending order starting from P1 comparing the distances d of the boundary points to the value Rs*(1+ε) until it encounters a point which has a smaller distance (labeled P2). The algorithm continues to traverse the boundary point set in ascending order if the angle ζ keeps decreasing to find the last point of the aneurysm neck. (Otherwise, the last point of the aneurysm neck is the last point with a distance larger than or equal to Rs*(1+ε)). In step 35 each boundary point is classified either as part of the artery wall or the aneurysm.

4. Geometrical 3D Parameters Characterizing the Aneurysm

After the search algorithm has successfully ended, it has identified the first and the last aneurysm boundary point. In steps 36 and 37 the area of the aneurysm neck is estimated. The area of the aneurysm neck is estimated as the sum over all neck angles multiplied by the average vessel radius and by the slice thickness (see also step 28).

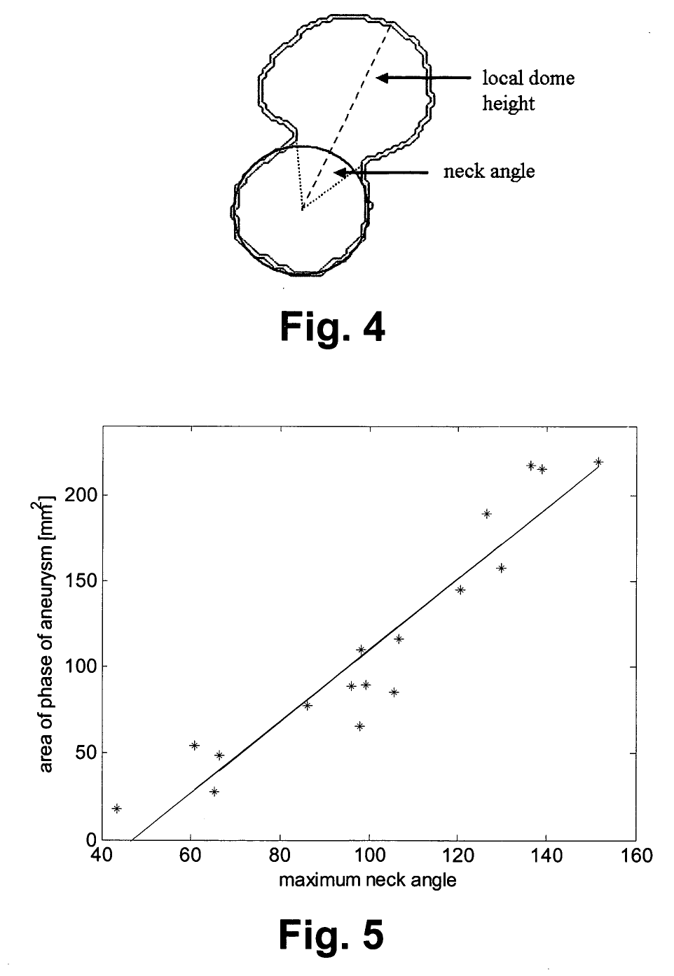

In step 36, the difference between their angles ζ is defined as the neck angle in this cross section, as depicted in FIG. 4. As depicted in FIG. 4, the fuzzy lines represent the lumen boundary, the solid line represents the reconstructed artery, the dotted lines represent the aneurysm neck, and the dashed line represents the local dome height. The maximum neck angle is the maximum over all neck angles from all cross sections and the sum of the neck angles is sum over all neck angles from all cross sections.

Referring now to FIG. 3, the local dome height of the aneurysm in each cross section is the maximum distance of the aneurysm boundary points from the vessel center. In FIG. 3 the distance d from center is plotted versus the polar angle. The black squares denote the first and last boundary points of the aneurysm neck found by the algorithm. The dome height of the aneurysm is the maximum over all local dome heights minus the average vessel radius. This definition is analogous to the dome height that is derived from the 2D DSA images. The neck length of the aneurysm is calculated by multiplying the number of cross sections containing the aneurysm neck with the slice thickness. The average vessel radius is the average over all cross sections.

5. 3D Visualization of Aneurysm and Reconstructed Artery

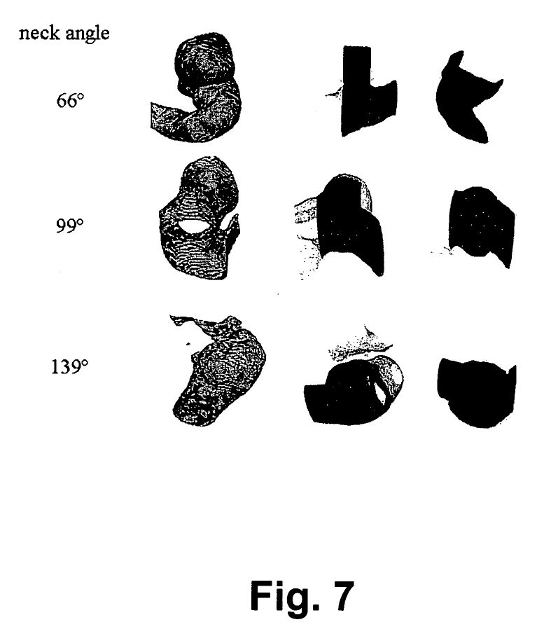

In step 29 the visualization toolkit (VTK) creates surface models of the boundary sets and the reconstructed artery. FIG. 7 illustrates four representative cases of these surface models. The colors gray and blue are the vessel and aneurysm wall, and the color green is the reconstructed artery.

The preferred embodiment of the apparatus used to perform the above-method includes a computer platform that is Java compatible, because the preferred embodiment of the method of the present invention is software program written in Java and C++ programming languages. More specifically, the embodiment is a personal computer (PC) with Microsoft Windows 2000 operating system. However, the program may be compiled and executed on any workstation that is capable of running programs written in Java with graphics. It is also possible to transfer the algorithm into any other programming language as it consists of calculations which can be implemented in almost any computer language. In this case a means has to be provided to indentify the boundary pixels.

The method of the present invention was used to visualize aneurysms in cases for evaluation of the method of the present invention. For the 17 investigated cases, Table 1 lists the results for the average vessel radius, the area of the aneurysm neck, the dome height and the maximum neck angle.

TABLE 1
Average vessel radius, area of the aneurysm
neck, dome height and maxium neck angle

| | average | | | | |
|---|---|---|---|---|---|
| | vessel | aneurysm | | maximum | neck |
| | radius | neck area | dome height | neck angle | length |
| case | [mm] | [mm²] | [mm] | [degree] | [mm] |
| 1 | 2.3 | 5.4 | 6.9 | 66.3 | 2.4 |
| 2 | 2.1 | 9.8 | 4.2 | 99.3 | 3.5 |
| 3 | 1.3 | 3.0 | 4.3 | 65.3 | 2.6 |
| 4 | 2.2 | 23.6 | 5.0 | 138.9 | 5.6 |
| 5 | 2.2 | 24.1 | 3.3 | 151.6 | 5.4 |
| 6 | 2.3 | 23.8 | 6.9 | 136.4 | 4.9 |
| 7 | 2.1 | 9.7 | 4.9 | 95.9 | 3.4 |
| 8 | 1.8 | 7.2 | 4.6 | 97.8 | 3.6 |
| 9 | 2.18 | 17.3 | 8.2 | 129.8 | 4.5 |
| 10 | 2.3 | 20.8 | 8.0 | 126.5 | 5.2 |
| 11 | 2.6 | 2.0 | 7.5 | 43.6 | 1.2 |
| 12 | 1.7 | 12.7 | 4.6 | 106.9 | 4.7 |
| 13 | 2.4 | 12.0 | 8.4 | 98.3 | 3.6 |
| 14 | 2.3 | 6.0 | 27.7 | 61.0 | 2.7 |
| 15 | 2.0 | 15.9 | 6.0 | 120.6 | 4.6 |
| 16 | 2.2 | 9.4 | 2.3 | 105.6 | 3.5 |
| 17 | 2.2 | 8.5 | 5.0 | 86.0 | 3.3 |

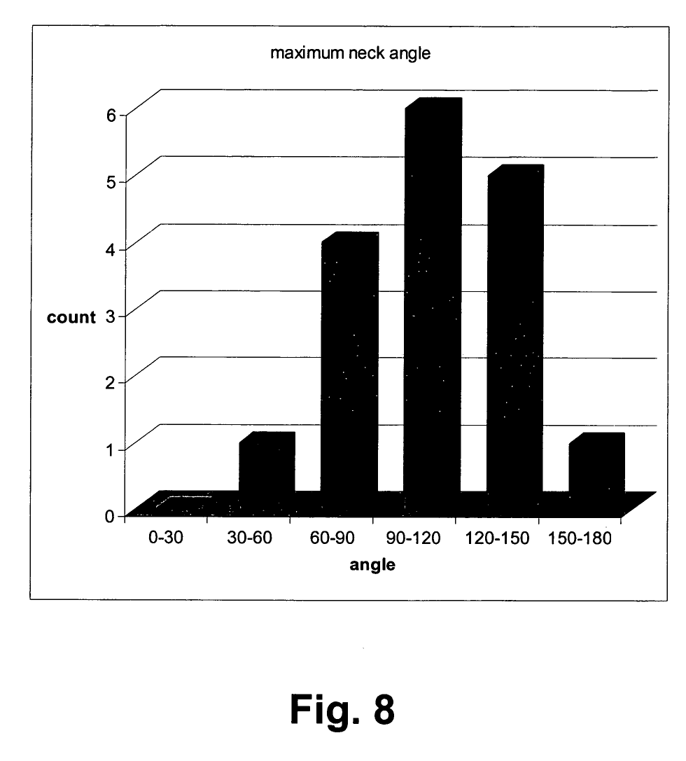

The statistical mean for the average vessel radius is 2.1±0.3 mm which is a typical value for intracranial arteries. In FIG. 8, the distribution of the maximum neck angle is shown. For the investigated aneurysms, the mean of this distribution was found to be 102±30 degrees. Table 2 shows the correlations between the investigated parameters.

TABLE 2
Correlations Between Investigated Parameters

| | Aneurysm neck | Dome | Maximum neck | Neck |
|---|---|---|---|---|
| | area | height | angle | length |
| Average vessel | 0.21 | 0.26 | 0.03− | −0.06 |
| radius | | | | |
| Aneurysm neck | — | −0.18 | 0.95 | 0.93 |
| area | | | | |
| Dome height | — | — | −0.35 | −0.25 |
| Maximum neck | — | — | — | 0.96 |
| angle | | | | |

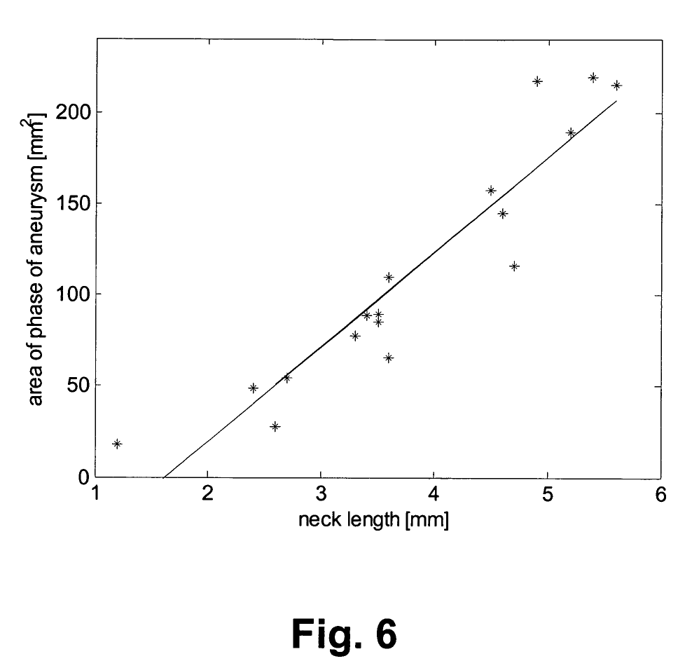

Referring now to both Tables 1 and 2, and to FIGS. 5 and 6, strong correlations (correlation coefficient>=0.93) are found between the maximum neck angle, the neck length and the area of the aneurysm neck. These correlations are interpreted to reflect the circular shape of the aneurysm neck. No significant correlations between the other parameters were found.

An alternate embodiment of the present invention uses a software package called “Inspace” on a Siemens Leonardo workstation. Angiographic data were obtained with a bi-plane C-arm system (Axiom Artis; Siemens Medical System, Erlangen, Germany) using commercially available hardware and software. The algorithm used for creating a virtual image of the full extent of a stent in the parent artery was implemented as a plug-in for the 3D image post-processing software package Inspace on the Siemens Leonardo workstation (version 2004B). All images were analyzed retrospectively after treatment had been completed.

After a volume of interest containing the aneurysm and proximal and distal segments of the healthy parent artery had been chosen by manually clipping the 3D-DSA data, the centerline of the normal segments of the parent artery, proximal and distal to the aneurysm, was computed using image post-processing skeletonization algorithms. From these centerline segments the centerline of the parent artery across the aneurysm ostium was then interpolated. Next, a set of contiguous 2D cross sections (cut planes) (approx. 0.1 mm thickness) containing the entire volume of the normal parent artery segments and the aneurysm and oriented perpendicular to the interpolated centerline was obtained. Then, for each cross section containing a portion of the aneurysm, the corresponding radius of the virtual parent artery was linearly interpolated using the radii measured at the normal proximal and distal segments of the parent artery.

The resulting reconstruction was then projected for analysis in three different views: a) as a series of 2D cross sections, b) as a 3D cut surface reconstruction (clipped by a cut plane so as to allow inspection of the inside of the aneurysm), and c) as a 3D surface rendered volume.

Referring now to FIG. 9c, an alternate method for the first nine steps of the method of the present invention is shown.

1. Interpolation of the Centerline of the Parent Artery

In Step 41, the Siemens Leonardo workstation provides the ability to create a 3D volume reconstruction of the acquired 3D DSA data. This ability is realized within the Inspace software (part of the Leonardo workstation). In step 42, the clipping function of Inspace is used to clip the 3D DSA data down to a volume of interest, which contains the aneurysm together with adjacent distal and proximal parts of the healthy parent artery. In step 43, the points of the centerlines of these proximal and distal vessel segments adjacent to aneurysm, as well as the boundary points of the vessel and the aneurysm, are determined by a post-processing skeletonization algorithm (provided by Siemens Medical Systems). In step 44, a Hermite polynom as the best approximation to the points of the centerlines calculated in step 43 is determined using a single value decomposition algorithm (SVD). This Hermite polynom is the interpolated centerline of the parent artery across the lateral extension of the aneurysm neck as well as of the distal and proximal vessel segments that were included in this calculation. The user can chose the length of the distal and proximal vessel segments to be included.

2. Reconstruction of the Virtual Parent Artery across the Aneurysm Neck (Virtual Stent)

In step 45, consecutive cross sections of approximately 0.1 mm thickness are calculated perpendicular to the interpolated vessel centerline (based on software code provided by Siemens). In the distal and proximal vessel sections, the radii of the parent artery are then determined for each cross section as the center of mass of the boundary points. The boundary points were calculated during skeletonization in step 42, and are now stored in an ordered fashion in a circular buffer for each cross section. In step 46, an average radius for the proximal segment and an average radius for the distal segment are then calculated. The number of cross sections used in this average calculation is typically small (4-10) and can be adjusted by the user. By linear interpolation between these two average radii, the radius of the virtual parent artery is then found for each cross section. This process yields the reconstruction of the parent artery without aneurysm, which we call virtual parent artery or virtual stent.

3. Determination of Neck Angle

In step 47, the neck angle in each cross section is then determined as follows. For each cross section, the corresponding set of boundary points is traversed in an ordered fashion. A boundary point that has a distance from the center of the reconstructed virtual artery larger than a certain percentage of its radius (user determined, typical values range from 10%-30%), marks the startpoint of a pocket. The endpoint of a pocket is reached, when the distance of a boundary point is again smaller than this certain percentage of the radius. This algorithm can yield 1) no pocket, i.e. the neck angle for this cross section is zero, 2) no endpoint for the first pocket, i.e. the neck angle for this cross section is 360 degrees, 3) exactly one pocket, the neckangle is then the angle difference between the vectors from the center of the virtual artery to the endpoints and to the startpoint, 4) more than one pocket. In the last case, the angle difference between startpoint and endpoint is calculated for each pocket and the neck angle is chosen from the pocket with the largest area (approximated by the product between the angle difference and the maximum distance between the vessel center and a boundary point contained in that pocket).

4. Calculation of 3D Parameters of the Aneurysm

The 3D parameters of the aneurysm are determined in step 48. The maximum neck angle is the maximum over all neck angles from all cross sections. The local dome height of the aneurysm in each cross section is the maximum distance of the aneurysm boundary points from the vessel center minus the radius of the virtual artery. The dome height of the aneurysm is the maximum over all local dome heights. The neck length of the aneurysm is calculated by multiplying the number of cross sections containing the aneurysm neck with the slice thickness. The start and end cross section have to be determined by the user by inspection. In order to minimize noise contributions, the neck angle is first interpolated (using Hermite Interpolation). The average vessel radius is the average over all cross sections. The area of the aneurysm neck is calculated by multiplying the arc length (determined by the neck angle and the vessel radius) in each cross section with the thickness of the cross section and summing over all cross sections.

5. Display of Results

The results are displayed in step 49. A four-panel view (based on Inspace software code provided by Siemens) is utilized. In the upper left panel, a cross section is displayed together with the boundary points, the reconstructed virtual artery (circle), and the neck angle. A scroll bar on the right of the panel allows the user to scroll through all the cross sections. The lower left panel displays the numeric results for the 3D parameters of the aneurysm together with a 2D plot of the neck angle or the local dome height (chosen by the user). The upper right panel shows a 3D surface reconstruction of the lumen boundary (vessel and aneurysm) together with the reconstructed virtual artery (or virtual stent). The lower right panel displays the original 3D volume reconstruction together with the reconstructed virtual artery, the interpolated centerline, and the cross section displayed in the upper left panel.

In summary, the steps of the alternate embodiment are:
a. manually clipping a 3D-DSA data to obtain a volume of interest containing the aneurysm and proximal and distal segments of a healthy parent artery;
b. computing the centerline of the normal segments of the parent artery, proximal and distal to the aneurysm, using image post-processing skeletonization algorithms;
c. from these centerline segments, interpolating the centerline of the parent artery across the aneurysm ostium;
d. obtaining a set of contiguous 2D cross sections (cut planes) (approx. 0.1 mm thickness) containing the entire volume of the normal parent artery segments and the aneurysm, and oriented perpendicular to the interpolated centerline;
e. for each cross section containing a portion of the aneurysm, linearly interpolating the corresponding radius of the virtual parent artery, using the radii measured at the normal proximal and distal segments of the parent artery; and
f. projecting the resulting reconstruction for analysis in three different views: a) as a series of 2D cross sections, b) as a 3D cut surface reconstruction (clipped by a cut plane so as to allow inspection of the inside of the aneurysm), and c) as a 3D surface rendered volume.

Using the method of the present invention, the morphology of two aneurysms that were treated with stent assisted coiling was assessed. One was a paraophthalmic aneurysm having a sidewall geometry (case 1) the other was a carotid bifurcation aneurysm (case 2). Each figure displays: a) 2D DSA projection images (AP and lateral view) before treatment and a snapshot of the 3D-DSA surface volume reconstruction, and b) a selected cross section, a 3D cut surface volume reconstruction, and a 3D surface volume overlaid with the virtual reconstructed artery. For comparison, a post-treatment 2D-DSA (AP and lateral projections) are shown in c.

Case 1: Wide Neck Paraophthalmic Aneurysm

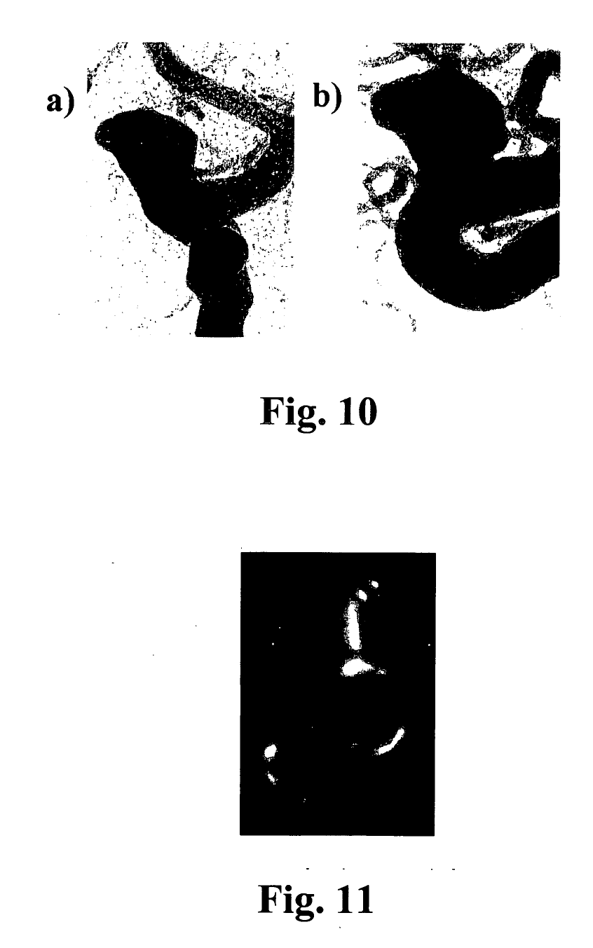

Referring now to FIGS. 10a and 10b, the pre-treatment AP and lateral 2D-DSA projection images show the course and size of the parent artery, the aneurysm size, and the neck length.

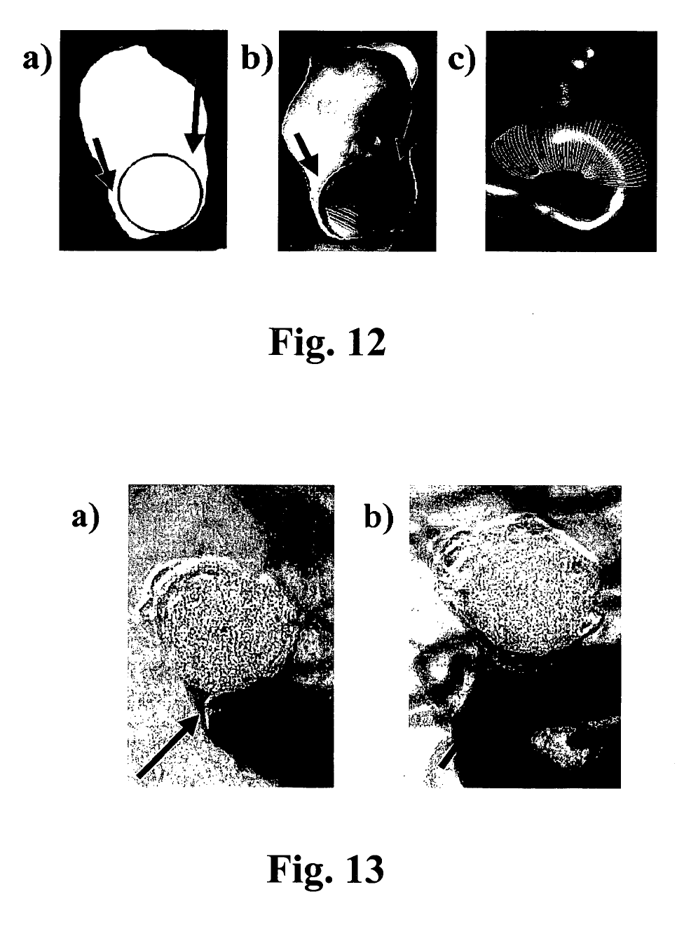

Referring now to FIG. 11, the 3D-DSA surface volume reconstruction shows to better advantage the expansion and irregularity of the portion of the ventral wall of the internal carotid artery from which the aneurysm arises. Neither of these show, however, that the aneurysm ostium involves at least 180 degrees of the parent artery circumference. Referring now to FIGS. 12a and 12b, this feature is clearly shown in the cut-plane section and the cut-surface volume reconstruction. Both of these also demonstrate well the “pockets” of the aneurysm that lie outside of the boundaries of the virtual stent. The blue circles mark the location of the virtual stent in each picture. The green arrows depict “pockets” or cul-de-sacs around the virtually reconstructed artery. Referring now to FIG. 12c, the fit of the virtual stent can be verified in the 3D DSA surface volume reconstruction.

Referring now to FIGS. 13a and 13b, coil loops in the “pocket” along the medial side of the aneurysm appear to lie within the parent artery (see where the arrows point) on the AP and lateral post treatment 2D-DSA projection images. During treatment it was not possible to achieve a working projection that separated clearly this component of the aneurysm from the parent artery.

Case 2: Wide neck carotid bifurcation aneurysm

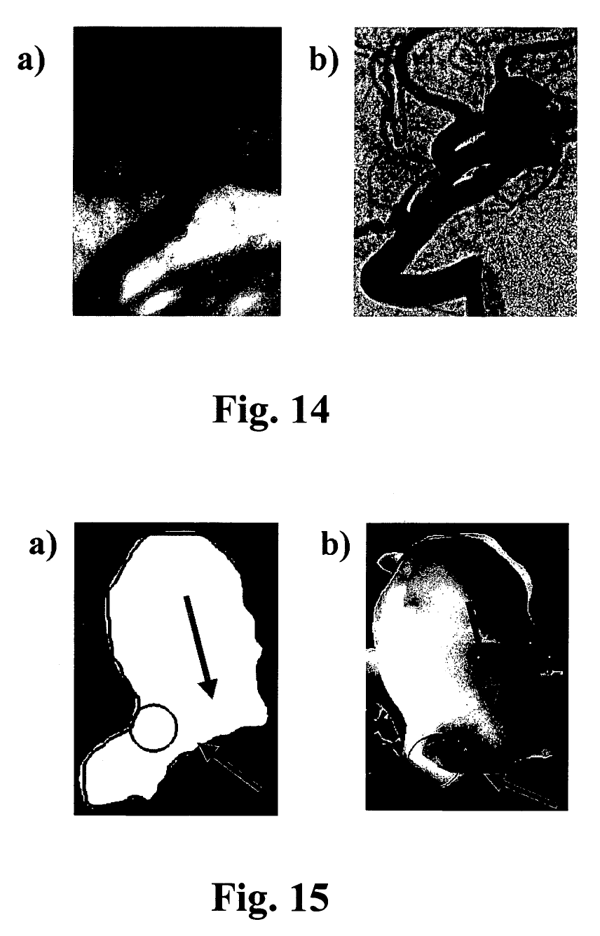

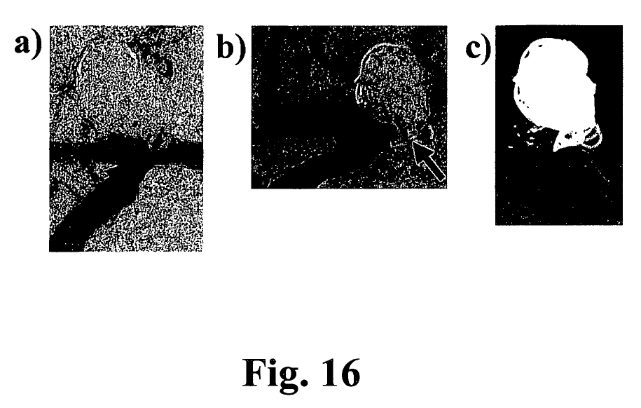

Referring now to FIGS. 14a and 14b, the AP and lateral pre-treatment 2D-DSA projection images show clearly the course of the parent artery, the aneurysm size, and the neck length. Referring now to FIGS. 15a and 15b, the cut-plane section and cut-surface volume reconstruction in a lateral projection and with a circle inserted to show the location and size of the interpolated normal parent artery demonstrate the extension of the aneurysm ostium posterior to the boundary of the parent artery. The circle in a) marks the position of the virtual stent in this cut-plane. The arrows demonstrate the extension of the aneurysm ostium posterior to the boundary of the parent artery. Referring now to FIGS. 16a, 16b, and 16c, the Post-treatment AP and lateral 2D-DSA images show coils that appear to be within the parent artery (see where the arrows point). Comparing these with the cut-plane section and cut-surface volume reconstruction shows that these are, in fact, outside of the boundaries of the stent, and are not compromising the parent artery.

The method of the present invention does not model the stent deployment by highly sophisticated means such as finite elements, but rather assumes that a stent deployed so that it passes from a proximal segment of normal artery, across an aneurysm ostium and into a distal segment of parent artery, will reconstruct the arterial boundaries to duplicate those of a normal artery. Looking at the 3D-DSA volume reconstructions for the two examples shown, one can see that the excellent fit of the reconstructed artery from which the aneurysm arises, with the normal proximal and distal segments, indicate that, in these two examples, this assumption is valid.

In addition to the utility of the method of the present invention in supplying endovascular therapists with an improved reconstruction of the geometry and morphology of aneurysms for use in pretreatment planning, the invention may also be useful for many other clinical applications. One such example is that it can easily be implemented in the current technology that creates the three-dimensional surface models of the aneurysm.

The advantage of the method of the present invention is that it creates a semi-automated three-dimensional classification of the geometry of lateral and saccular intracranial aneurysms using the information provided by 3D DSA. The method interpolates the artery segment across the length of the aneurysm neck, and therefore allows for the creation of a three-dimensional surface reconstruction of the aneurysm together with the (virtual) reconstructed artery. The method also provides a three-dimensional characterization of the geometry of the aneurysm by quantifying not only commonly used geometric parameters (such as neck length, dome height and dome-to-neck ratio), but it also determines the center and the radius of the parent artery, the maximum neck angle of the aneurysm in cross sections perpendicular to the axis of the parent artery, a measure for the area of the aneurysm, and the lateral neck length. These three-dimensional parameters can then be correlated with treatment outcomes.

The method and apparatus of the present invention overcome the shortcomings of the prior art by supplying endovascular therapists with an enhanced reconstruction of the geometry and morphology of intracranial aneurysms for purposes of pretreatment planning.

Though the invention has been disclosed with reference to preferred embodiments, it will be understood by those skilled in the art that various changes in form and detail may be made without departing from the spirit and scope of the invention.

What is claimed is:

1. A method of creating a surface model of an intracranial aneurysm in an artery having a lumen, the aneurysm having a neck and a dome, the method comprising the steps of:
a. determining a center and radius of the artery over the lateral extension of the aneurysm;
b. determining the boundary points that mark the boundary between the aneurysm neck and the artery;
c. determining the angle of the aneurysm neck with respect to the artery, for various cross sections of the neck;
d. determining the length of the neck;
e. determining the height of the dome;
f. estimating the area of the neck; and
g. creating the surface model of the intracranial aneurysm in the artery, using the results from the previous steps.

2. The method of claim 1, wherein the step of determining a center and radius of the artery comprises the steps of:
a. constructing a 3D maximum intensity projection to visualize the orientation of the parent artery and the aneurysm; and
b. creating a set of 2D cross sections oriented approximately perpendicular to the axis of the artery.
c. sorting the 2D cross sections to discard the neck cross sections; and
d. iteratively determining the center and radius of the artery for all remaining cross sections over the lateral extension of the aneurysm.

3. The method of claim 2, wherein the step of determining the angle of the aneurysm neck comprises the steps of:
a. identifying the first and the last aneurysm boundary points; and
b. determining the angle of the aneurysm neck by taking the difference between the angles of the first and the last aneurysm boundary points.

4. The method of claim 3, wherein the step of determining the area of the aneurysm neck comprises the steps of:
a. summing all the neck angles to create a sum; and
b. multiplying the sum by the average artery radius and by the thickness of the 2D cross sections.

5. A computer system configured in any manner for performing a method of creating a surface model of an intracranial aneurysm in an artery having a lumen, the aneurysm having a neck and a dome, the computer system comprising:
a. means for determining a center and radius of the artery over the lateral extension of the aneurysm;
b. means for determining the boundary points that mark the boundary between the aneurysm neck and the artery;
c. means for determining the angle of the aneurysm neck with respect to the artery, for various cross sections of the neck;
d. means for determining the length of the neck;
e. means for determining the height of the dome;
f. means for estimating the area of the neck; and
g. means for creating the surface model of the intracranial aneurysm in the artery, using the results from the previous steps.

6. A computer-readable storage medium encoded with executable instructions, representing a computer program, to cause a computer to perform a method of creating a surface model of an intracranial aneurysm in an artery having a lumen, the aneurysm having a neck and a dome, the method comprising the steps of:
a. determining a center and radius of the artery over the lateral extension of the aneurysm;
b. determining the boundary points that mark the boundary between the aneurysm neck and the artery;
c. determining the angle of the aneurysm neck with respect to the artery, for various cross sections of the neck;
d. determining the length of the neck;
e. determining the height of the dome;
f. estimating the area of the neck; and
g. creating the surface model of the intracranial aneurysm in the artery, using the results from the previous steps.

7. A method of allowing visualization of a virtual stent deployed across an aneurysm ostium, the method comprising the steps of:
a. manually clipping a 3D-DSA data to obtain a volume of interest containing the aneurysm and proximal and distal segments of a healthy parent artery;
b. computing the centerline of the normal segments of the parent artery, proximal and distal to the aneurysm;
c. from these centerline segments, interpolating the centerline of the parent artery across the aneurysm ostium;
d. obtaining a set of contiguous 2D cross sections containing the entire volume of the normal parent artery segments and the aneurysm, and oriented perpendicular to the interpolated centerline;
e. for each cross section containing a portion of the aneurysm, linearly interpolating the corresponding radius of the virtual parent artery; and
f. projecting the resulting reconstruction for analysis.

8. The method of claim 7, wherein the step of computing the centerline of the normal segments uses image post-processing skeletonization algorithms.

9. The method of claim 8, wherein the step of linearly interpolating the corresponding radius of the virtual parent artery uses the radii measured at the normal proximal and distal segments of the parent artery.

10. The method of claim 9, wherein the step of projecting the resulting reconstruction for analysis is done in three different views:
a. as a series of 2D cross sections;
b. as a 3D cut surface reconstruction; and
c. as a 3D surface rendered volume.

11. The method of claim 10, wherein the projection as a 3D cut surface reconstruction is clipped by a cut plane so as to allow inspection of the inside of the aneurysm.

12. A computer system configured in any manner for performing a method of allowing visualization of a virtual stent deployed across an aneurysm ostium, the computer system comprising:
a. means for manually clipping a 3D-DSA data to obtain a volume of interest containing the aneurysm and proximal and distal segments of a healthy parent artery;
b. means for computing the centerline of the normal segments of the parent artery, proximal and distal to the aneurysm, using image post-processing skeletonization algorithms;
c. means for from these centerline segments, interpolating the centerline of the parent artery across the aneurysm ostium;
d. means for obtaining a set of contiguous 2D cross sections containing the entire volume of the normal parent artery segments and the aneurysm, and oriented perpendicular to the interpolated centerline;
e. means for linearly interpolating the corresponding radius of the virtual parent artery for each cross section containing a portion of the aneurysm and;
f. means for projecting the resulting reconstruction for analysis.

13. A computer-readable storage medium encoded with executable instructions, representing a computer program, to cause a computer to perform a method of allowing visualization of a virtual stent deployed across an aneurysm ostium, the method comprising the steps of:
a. manually clipping a 3D-DSA data to obtain a volume of interest containing the aneurysm and proximal and distal segments of a healthy parent artery;
b. computing the centerline of the normal segments of the parent artery, proximal and distal to the aneurysm;
c. from these centerline segments, interpolating the centerline of the parent artery across the aneurysm ostium;
d. obtaining a set of contiguous 2D cross sections containing the entire volume of the normal parent artery segments and the aneurysm, and oriented perpendicular to the interpolated centerline;
e. for each cross section containing a portion of the aneurysm, linearly interpolating the corresponding radius of the virtual parent artery; and
h. projecting the resulting reconstruction for analysis.
